(19) United States
(12) Patent Application Publication
(10) Pub. No.: US 2020/0123636 A1
(43) Pub. Date: Apr. 23, 2020

(54) MAGNESIUM ALLOY, BIODEGRADABLE IMPLANT AND METHOD FOR PRODUCING A BIODEGRADABLE IMPLANT

(71) Applicant: aap Implantate AG, Berlin (DE)

(72) Inventors: Amir Eliezer, Omer (IL); Bruke Seyoum Alemu, Berlin (DE)

(21) Appl. No.: 16/625,858

(22) PCT Filed: Jun. 26, 2018
(86) PCT No.: PCT/EP2018/067087

§ 371 Date: Dec. 23, 2019

(30) Foreign Application Priority Data
Jun. 27, 2017 (DE) 10 2017 114 254.1

(51) Int. Cl. C22C 23/04; C25D 11/02; C25D 11/30; A61L 31/02; A61L 31/08; A61L 31/14

(52) U.S. Cl. CPC C22C 23/04; C25D 11/026; C25D 11/30; A61L 31/022; A61L 2420/02; A61L 31/026; A61L 31/148; A61L 2400/18; A61L 31/086

(57) ABSTRACT

The invention relates to a magnesium alloy which comprises:
Zn: 0.5-2 wt %,
Mn: 0.2-1 wt %,
Ca: 0.1-2 wt %,
wherein the magnesium alloy comprises
0.6 wt % or more Mn or
0.6 wt % or more Ca,
between 0.5 wt % and 1.5 wt % Mn and Ca in sum,
and wherein Mg and impurities account for the remaining content in the alloy that is missing up to 100 wt %. The invention further relates to an implant of a calcium containing magnesium alloy, which is coated with a calcium phosphate layer. The invention further relates to a method of producing a biodegradable implant.

### TECHNICAL FIELD

The disclosure relates to a coated biodegradable implant. Further, the disclosure relates to a magnesium alloy and to a method of producing a biodegradable implant.

### BACKGROUND

Biodegradable implants that corrode after implantation are known. In particular, magnesium and magnesium alloy implants that corrode after implantation can be used for treating bone fractures.

Document EP 1 840 235 B1 describes a magnesium alloy and a method for producing it. This alloy comprises yttrium. Typically, such an alloy comprises further rare earth elements.

Document WO 2014/145672 A1 describes a microalloyed magnesium material, which comprises zinc, calcium, and manganese.

It is a problem for many applications that magnesium alloy-based implants corrode quickly. This may result in reducing the necessary mechanical properties quickly. The corrosion process also results in gas formations. However, large gas formations shall be avoided and the corrosion rate of the implant should be controlled, e.g., an implant with small, fragile structures should not corrode as quickly as an implant with more massive structures.

### SUMMARY

It is an object of the disclosure to provide a biodegradable implant based on a magnesium alloy with a controlled corrosion rate and with sophisticated properties with respect to the ingrowth of bone structure. The disclosure relates to implants with or without a bioactive coating.

The object of the disclosure is achieved by providing a magnesium alloy for a biodegradable implant, by a coated biodegradable implant and by a method for producing a biodegradable implant. Specific embodiments and refinements are subject of the description, and of the drawings.

A biodegradable implant comprises a body of a magnesium alloy, wherein said magnesium alloy comprises Ca, and wherein said magnesium alloy is coated with a coating which comprises Ca.

Besides a coated biodegradable implant, the disclosure relates to a magnesium alloy for a biodegradable implant having the following composition:

### Zn: 0.5-2 wt %,

### Mn: 0.2-1 wt %,

### Ca: 0.1-2 wt %,

wherein the magnesium alloy comprises
0.6 wt % or more Mn or
0.6 wt % or more Ca,
between 0.5 wt % and 1.5 wt % Mn and Ca in sum,
and wherein Mg and impurities account for the remaining content in the alloy that is missing up to 100 wt %.

By providing a biodegradable magnesium alloy having the claimed composition, it is possible to provide a material with good mechanical properties. In further, the corrosion rate can be influenced by varying the three components zinc, manganese, and calcium in the claimed ranges.

Calcium seems to promote the ingrowth of natural bone tissue. Zinc and manganese seem to stimulate the mechanical strength of the implant. All three components seem to interact. The inventors found out that the alloy should comprise at least 0.6 wt % of manganese or calcium, but not more than 1.5 wt % of manganese and calcium in sum.

With the exception of impurities, the claimed alloy composition does not comprise any further components.

In particular, the claimed magnesium alloy is free of yttrium, and, preferably, also free of any other rare earth elements.

The magnesium alloy can be produced, e.g., by casting. In particular, the alloy can be produced by melting pure magnesium with calcium, manganese and zinc in order to cast a very pure alloy composition with the claimed components.

However, the alloy can also be cast into strands, which are drawn in order to increase the mechanical properties.

Preferably, the alloy is used for producing an implant by using abrasive methods, e.g., by cutting, milling, turning, and grinding a block of alloy to the desired shape of an implant body.

The implant body is preferably a non-porous body.

According to a preferred embodiment of the invention, the magnesium alloy has the following composition:

Zn: 1-2 wt %, preferably 1.2-1.8 wt %, particular preferred 1.2-1.8 wt %,
Mn: 0.3-1 wt %, preferably 0.3-0.9 wt %, particular preferred 0.6-0.8 wt %, and/or
Ca: 0.2-1 wt %, preferably 0.3-0.7 wt %, particular preferred 0.2-0.4 wt %.

Preferably, the alloy comprises between 0.8 wt % and 1.2 wt % manganese and calcium in sum.

The alloy comprises preferably less than 0.4 wt %, in particular less than 0.1 wt % impurities.

Possible impurities are in particular silicone, iron, copper and nickel. However, the implant preferably comprises less than 0.01 wt % silicone and/or iron, less than 0.005 wt % copper and/or less than 0.001 wt % nickel.

The invention further relates to the use of the magnesium alloy to produce a medical device, in particular to produce an implant.

The implant can be provided with or without a bioactive coating, e.g. a coating comprising calcium phosphate.

According to an embodiment of the invention, the magnesium alloy is produced by using an extrusion process.

In particular, according to the invention, an ingot, in particular, a cylinder-shaped ingot, is produced by using a casting process.

Then, this ingot is extruded, preferably in two steps.

For example, the ingot is extruded to a rod in a first step to its 0.15 to 0.5 diameter. The extruded rod is then extruded in a second step to its 0.1 to 0.5 diameter.

By using an extrusion process, the mechanical strength of the alloy can be improved, in particular an increased tensile strength, at least more than 20%, preferably at least more than 40%, can be achieved.

Preferably, the alloy has a tensile strength of more than 250 N/mm2, in particular a tensile strength between 250 and 600 N/mm2 (measured according to DIN 50125 (revision 2009-07)).

The alloy has a preferably an elongation at fracture of more than 8%, particular preferred of more than 10%, in particular of 8-20%.

In addition, the invention relates to a biodegradable implant comprising a body of a magnesium alloy. Preferably, the body consists of a magnesium alloy as described above.

The magnesium alloy comprises calcium, in particular at least 0.1 wt % calcium, preferably at least 0.2 wt % calcium.

According to one embodiment, the magnesium alloy comprises 0.2-3 wt % Mn, 0.5-5 wt % Zn and/or 0.1-3 wt % Ca and/or a ratio of Ca/Mn between 3:1 and 1:3 and/or a ratio of Zn to the sum of Ca and Mn between 2:1 and 1:1.

The magnesium alloy, respectively the body of the magnesium alloy, is covered with a coating that comprises calcium.

The combination of a calcium containing magnesium alloy with a coating that comprises calcium, in particular with calcium phosphate coating, results in an implant with slow corrosion in the initial period after implantation.

Additionally, the combination of a calcium-based coating and the calcium containing magnesium alloy results in a sophisticated ingrowth of bone tissue after the coating has been corroded.

The coating is preferably a continuous layer of the calcium phosphate, in particular of a hydroxyapatite.

The coating preferably comprises at least 5 wt %, particular preferred at least 30 wt % hydroxyapatite.

Preferably, the layer is a non-porous dense layer.

According to one embodiment, the coating has a thickness of 1 to 100 μm, preferably of 3 to 25 μm.

Preferably, coating also comprises magnesium, in particular magnesium in the form of magnesium oxide.

In particular, the magnesium content on the surface of the coating is more than 10 wt %, preferably more than 30 wt % and/or less than 70 wt %, preferably less than 60 wt %.

In particular, it is possible to form a calcium phosphate layer on the calcium-containing alloy, wherein the alloy gradually transitions to the coating. The magnesium content in the coating prevents the formation of flakes, which tend to peel off.

According to a preferred embodiment, the coating comprises at its surface 5-30 wt % Ca, 15-50 wt % 0 and/or 3-10 wt % P.

The coating can also comprise up to 10 wt % Si, which seems to serve as a bonding agent. In particular, the coating comprises at least 0.5 wt % Si.

The coating can comprise a ceramic-metal-matrix, wherein ceramic calcium phosphate particles are bonded in a metal matrix, in particular in a magnesium oxide matrix.

According to an embodiment, the calcium phosphate layer is applied by using a plasma electrolytic deposition method.

By using such a method, the implant body is immersed into an electrolyte bath. Preferably, the electrolyte bath comprises calcium phosphate particles, in particular hydroxyapatite particles.

According to an embodiment, a coating is applied by using an electrolyte, which comprises particles in two fractions of size.

Specifically, the electrolyte can comprise colloid-dispersed particles, in particular particles with an average size of 100 nm or less (measured with a TEM method), and a second larger fraction of particles, in particular microsize particles, with an average particle size of 10 μm to 100 μm (measured with a laser particle sizer).

Such a method of providing a calcium phosphate layer by using an electrolyte bath with two different fractions of particle size known from the document WO 2012/007181 A1.

However, the inventor discovered that there is a surprising interaction between the calcium containing alloy and a layer, which is applied by using the plasma electrolytic oxidation method.

In particular, by using the plasma electrolytic oxidation method, it is possible to apply a layer, wherein the magnesium alloy of the body of the implant gradually transitions to said coating.

Due to the high energy of a PEO coating, oxide particles, in particular magnesium oxide particles, are formed on the surface of the alloy.

These oxide particles form a layer system, wherein the intermediate layer between the pure alloy and the calcium phosphate layer comprises calcium phosphate and magnesium, in particular magnesium oxide. This intermediate layer preferably has a thickness between 0.5 and 2 μm.

The inventor also discovered that the layer which is applied by a plasma electrolytic oxidation process on a calcium containing magnesium alloy reduces the risk that the magnesium alloy corrodes quickly due to so-called “pitting”. A pitting typically occurs when the alloy corrodes below the calcium phosphate layer, thereby forming a hole, which results in the calcium phosphate layer peeling off at least in the area of the hole.

By applying a plasma electrolytic oxidation method, it is possible to cover the magnesium alloy with a dense calcium phosphate layer, thereby reducing the risk of an accelerated pitting.

The implant is embodied, e.g., as a screw or as a bone plate.

The electrolyte for the plasma electrolytic oxidation comprises according to an embodiment of the invention water glass and/or a dissolved phosphate, in particular sodium phosphate, preferably each in an amount of at least 0.1 wt % in the electrolyte.

Water glass seems to serve as a bonding agent and sodium phosphate seems to help to apply a more homogeneous layer.

The disclosure further relates to a method for producing a biodegradable implant, in particular an implant as described above. The implant body comprises a calcium containing magnesium alloy and is covered with a calcium phosphate coating by using a plasma electrolytic oxidation method.

In particular, the plasma electrolytic oxidation process is performed in an electrolytic bath, which comprises calcium phosphate particles, in particular hydroxyapatite particles.

With this method, it is possible to generate above described layer system comprising an intermediate layer so that the metal surface gradually transitions to the calcium phosphate layer.

The disclosure further relates to a method of controlling the corrosion of a biodegradable magnesium alloy, in particular an alloy as described before.

An alloy is produced which comprises 0.5-2 wt % Zn, 0.2-1 wt % Mn and 0.1-2 wt % Ca, wherein the corrosion rate is controlled by varying the content of Zn, Mn, and Ca.

The invention is based on the fact that the triangle of the components Zn, Mn, and Ca in the claimed ranges enables production of a magnesium alloy with good mechanical properties, wherein the corrosion rate can be controlled, depending on the application, by varying the amount of zinc, manganese, and calcium.

In particular, it is possible to provide a magnesium alloy having a corrosion rate in a 0.9% NaCl-solution at 37° C. of less than 3 mmpy (millimeters per year), preferably of less than 2 mmpy, at least one hour after immersing the alloy into the solution.

### Exemplary Alloy Compositions

Example 1:

### Zn: 1.5-1.7 wt %

### Mn: 0.7-0.8 wt %

### Ca: 0.25-0.35 wt %

Example 2:

### Zn: 1.5-1.7 wt %

### Mn: 0.2-0.4 wt %

### Ca: 0.6-0.8 wt %

Example 3:

### Zn: 1.2-1.4 wt %

### Mn: 0.7-0.8 wt %

### Ca: 0.25-0.35 wt %

All three examples preferably comprise less than 0.1 wt % impurities, in particular less than 0.01 wt % Si, less than 0.01 wt % Fe, less than 0.003 wt % Cu, less than 0.001 wt % Ni and/or less than 0.07 wt % other impurities.

It had been found out that the corrosion of the alloy according to Example 1 and 3 is significantly lower than the corrosion according to Example 2.

The alloy according to Example 2 is preferably used for applications, wherein a fast corrosion is intended or if the alloy is provided with a protective coating.

The alloy according to Example 1 and 3 can be used with or without coating.

### BRIEF DESCRIPTION OF THE DRAWINGS

FIG. 1 shows am implant.

FIG. 2 a schematic cross sectional view of an implant.

FIG. 3 is a table showing the corrosion rate of an implant.

FIG. 4 is a graph showing the corrosion rate of two implants according to the invention and of a comparative example.

FIG. 5 is an illustration of the controlled corrosion of the alloy.

FIG. 6 is a SEM image of the surface of a coated implant.

FIG. 7 is an EDS analysis of the surface of the coated implant.

FIGS. 8 and 9 are SEM images of a sliced coated implant.

FIG. 10 is an EDS analysis of the transition region between the alloy substrate and the coating.

FIG. 11-FIG. 14 are images of an element mapping of the surface region for the elements calcium, oxygen, phosphorous and magnesium.

### DETAILED DESCRIPTION

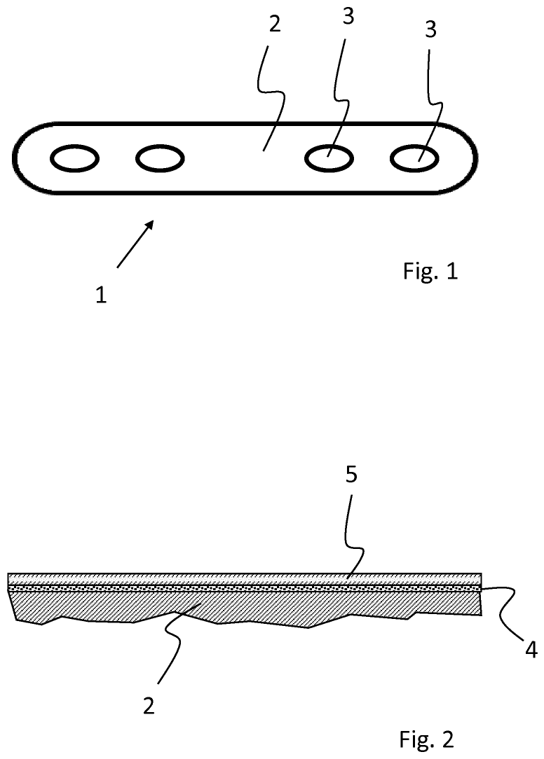

FIG. 1 is a schematic view of an implant 1. The implant 1 is formed in this embodiment as a bone plate, comprising a body 2 with holes 3 for inserting bone screws.

The implant body consists of a non-porous, massive magnesium alloy, preferably of an alloy composition, according to one of the examples. The material of the implant can also be densified, in particular by using an extrusion process.

The implant body 2 is covered with a calcium phosphate coating, which is shown in the schematic drawing according to FIG. 4.

The surface of the body 2 is coated by using the plasma electrolytic oxidation method.

The plasma electrolytic oxidation method is performed by introducing calcium phosphate, in particular hydroxyapatite particles, into an electrolytic bath, and by generating a plasma discharge at the surface of the implant body 2.

By using such a plasma electrolytic oxidation process, a calcium phosphate layer 5 is formed on the surface of the body.

Due to the high energy of the plasma discharge, an intermediate layer 4 forms, which comprises magnesium, in particular magnesium oxide, as well as calcium phosphate.

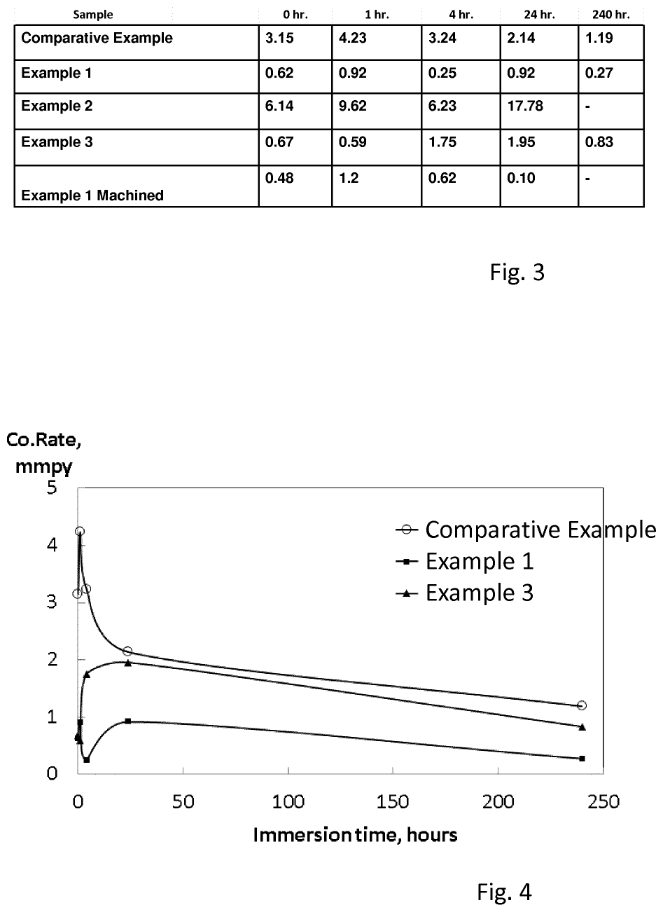

FIG. 3 is a table showing the corrosion rate (mmpy) of an implant, which is immersed into a 0.9% NaCl solution at a temperature of 37° C.

The table shows the corrosion rate of an alloy, according to Examples 1, 2, and 3. The corrosion rate of a machined implant made of the alloy according to Example 1 is also shown.

The table further shows a comparative example of another magnesium alloy. This alloy has a manganese content of 0.5%, a calcium content of 0.3% and a zinc content of 1.3%.

It can be seen that the corrosion rate of the alloy, according to Example 1 and Example 3, is lower than the corrosion rate of the comparative example, especially in the initial phase after implantation. In further, the corrosion rate is not significantly weakened by machining the alloy.

FIG. 4 is a graph showing the corrosion rate of above-mentioned comparative example and Example 1 and 3, dependent on the immersion time.

It can be seen that the corrosion rate of Example 1 is less than 1 mmpy over the entire period.

The corrosion rate of Example 3 is substantially higher and is similar to the comparative example after an immersion time of 24 hours.

However, the corrosion rate in the initial phase after immersion of the comparative example is substantially higher, in particular the corrosion rate reaches more than 3 mmpy.

This results in an increased gas bubble formation after implantation, which can be disadvantageous, depending on the application.

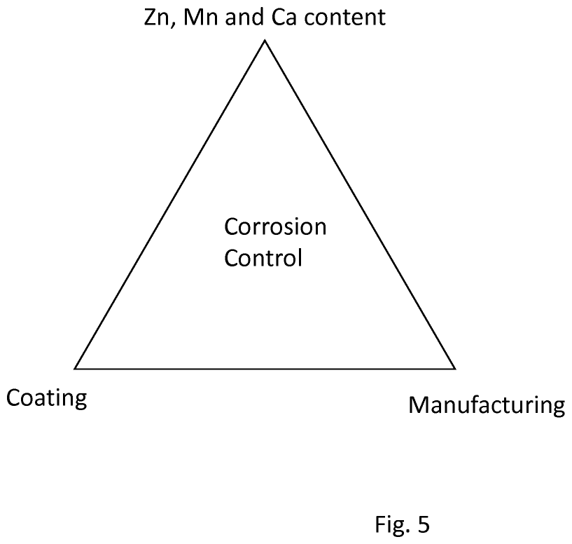

FIG. 5 shall illustrate that it is, according to the invention, possible to control the corrosion by at least varying the content of zinc, manganese, and calcium.

In further, the corrosion rate can also be controlled by the manufacturing method, in particular by using an at least two step extrusion process and by providing a coating, in particular a protective calcium phosphate coating.

With the invention-controlled degradation of biodegradable implants, a sophisticated ingrowth of bone tissue can be achieved.

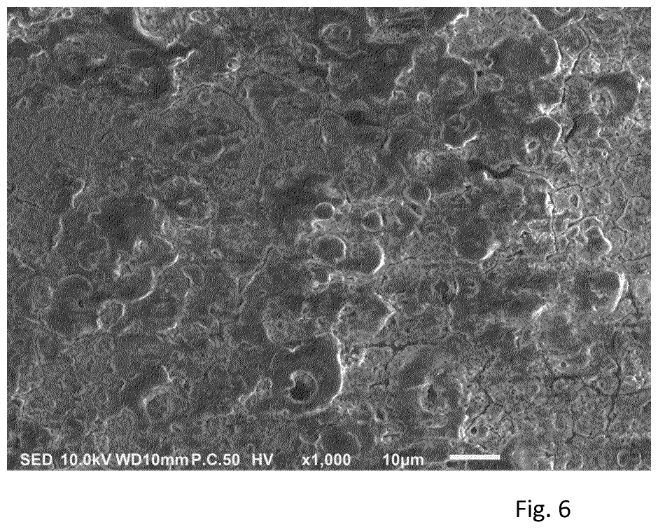

FIG. 6 is a SEM image of a calcium phosphate coating, which is applied onto a calcium containing magnesium alloy by using a plasma electrolytic oxidation method.

The coating consists of plateaus of a calcium phosphate layer, which are separated by meander-shaped grooves.

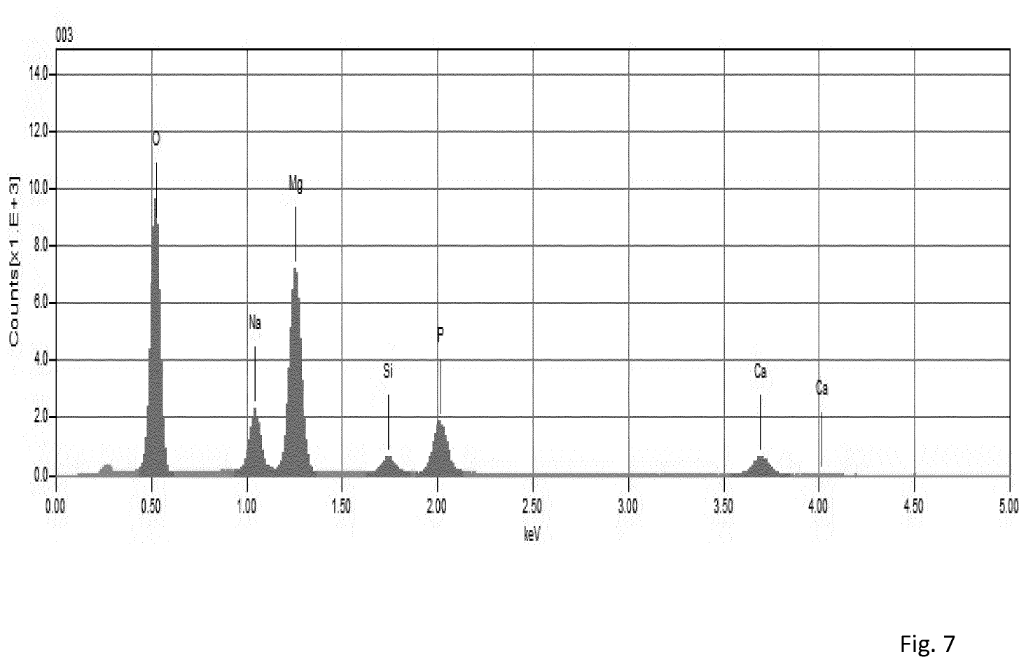

FIG. 7 is an EDS analysis of the surface of the coating. The coating comprises also a high amount of amount of magnesium oxide.

In this embodiment, the coating comprises 40-60 wt % Mg.

Preferably, the coating comprises at least at its surface 5-30 wt % Ca, 15-50 wt % 0 and 3-10 wt % P.

Since the used electrolyte also comprises water glass, the coating also comprises Si and Na.

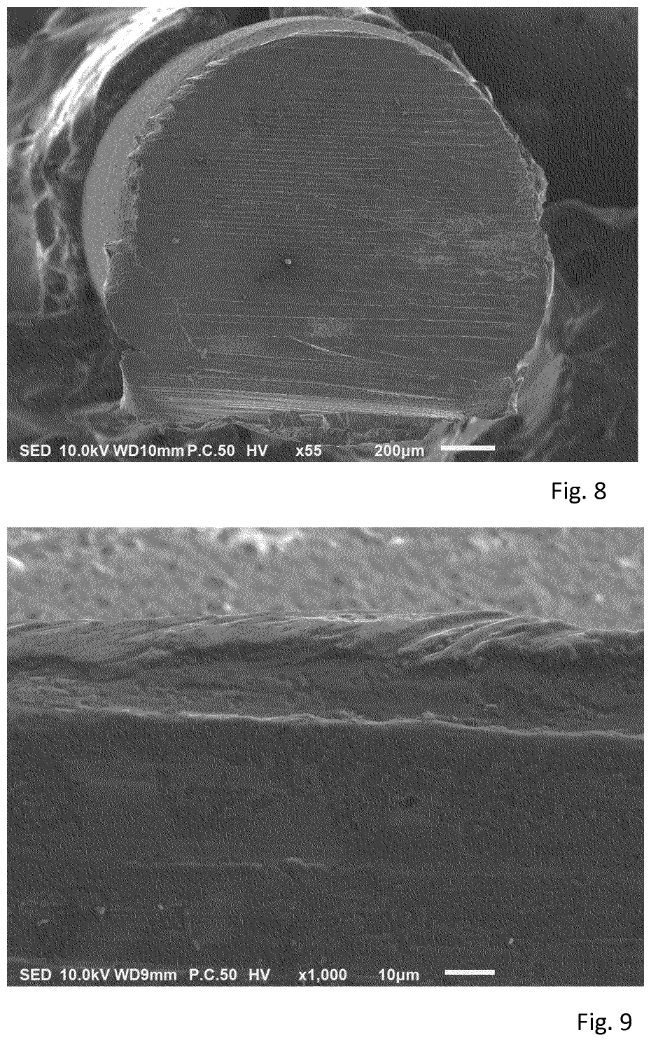

FIG. 8 is an SEM image of a sliced pin of a magnesium alloy comprising a calcium phosphate coating.

FIG. 9 is a detailed view of the coating. There is nearly no visible transition between the coating and the substrate.

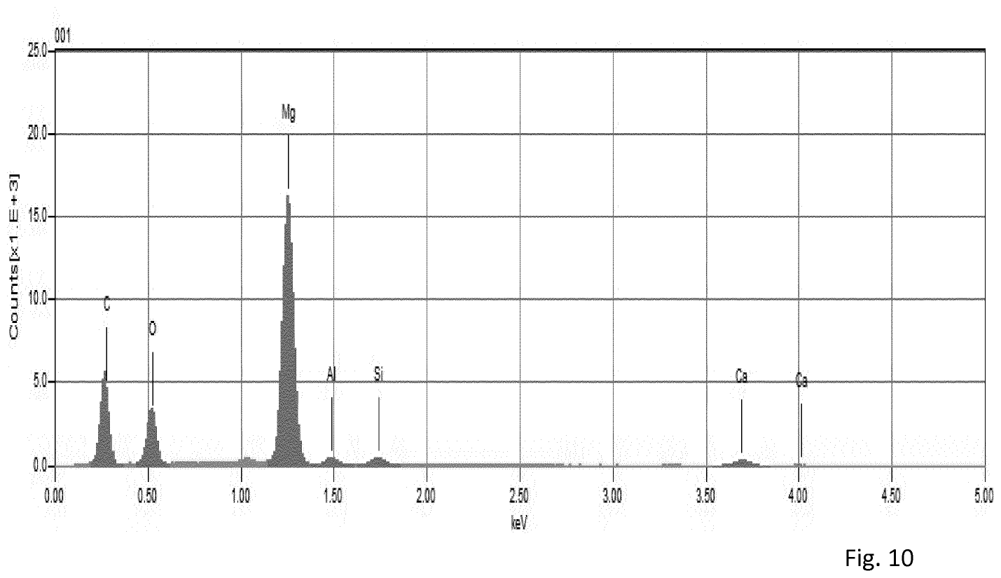

FIG. 10 is an EDS analysis of the transition region between the coating and the substrate.

In comparison to coating as shown in FIG. 7, the amount of calcium and oxygen is substantially lower.

However, it can be shown that the ca containing alloy gradually transitions to a coating which comprises MgO, Ca and P as main components.

In particular, the coating comprises hydroxyapatite, which is formed from the Ca and P containing electrolyte.

Without being bond to this theory, the Ca in the alloy enables the gradual transition, which results in an extreme strong bond of the coating.

The coating reduces the corrosion of the implant in the initial phase after insertion.

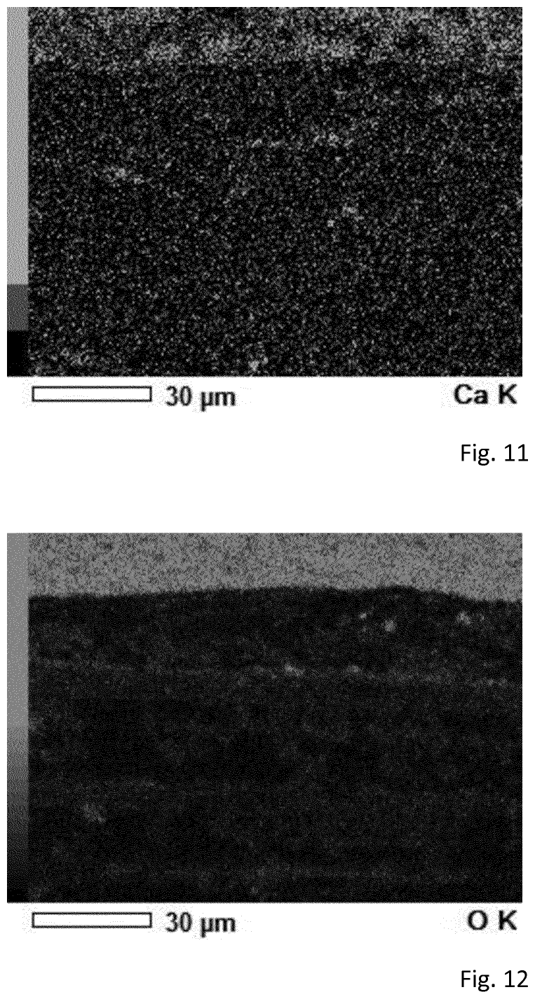

FIG. 11-FIG. 14 are images of an element mapping of the surface region of a coated implant for the elements calcium, oxygen, phosphorous and magnesium.

As shown in FIG. 11, the Ca content in the coating is substantially higher as in the alloy. However, also the alloy comprises Ca in order to enable a gradual transition of the substrate to the coating.

FIG. 12 shows that the alloy is substantially free of oxide. The coating has an oxygen content of at least 15 wt %, which is predominantly bonded as MgO.

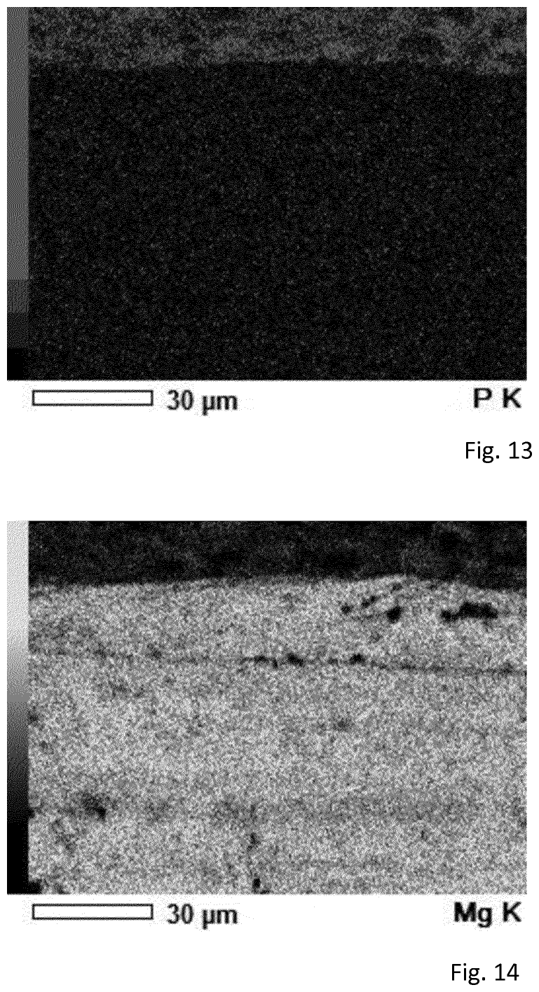

FIG. 13 shows that only the coating comprises phosphorous, in particular to be embodied as a hydroxyapatite containing layer.

FIG. 14 shows that also the coating comprises magnesium. However, since the magnesium in the coating is bonded predominantly in the form of MgO, also this oxide contributes the protective property of the coating.

1.-22. (canceled)

23. A magnesium alloy for a biodegradable implant, having the following composition:
Zn: 0.5-2 wt %,
Mn: 0.2-1 wt %,
Ca: 0.1-1.3 wt %,
wherein the magnesium alloy comprises
0.6 wt % or more Mn or
0.6 wt % or more Ca,
between 0.5 wt % and 1.5 wt % Mn and Ca in sum,
and wherein Mg and impurities account for the remaining content in the alloy that is missing up to 100 wt %.

24. The magnesium alloy of claim 23, having the following composition:
Zn: 1-2 wt %,
Mn: 0.3-1 wt %, and
Ca: 0.2-1 wt %.

25. The magnesium alloy of claim 23, having the following composition:
Zn: 1.2-1.8 wt %,
Mn: 0.3-0.9 wt %, and
Ca: 0.3-0.7 wt %.

26. The magnesium alloy of claim 23, having the following composition:
Zn: 1.2-1.8 wt %,
Mn: 0.6-0.8 wt %, and
Ca: 0.2-0.4 wt %.

27. The magnesium alloy of claim 23, wherein said magnesium alloy comprises between 0.8 wt % and 1.2 wt % Mn and Ca in sum.

28. The magnesium alloy of claim 23, wherein said alloy comprises less than 0.5 wt %.

29. The magnesium alloy of claim 23, wherein said alloy comprises less than 0.1 wt % impurities.

30. The magnesium alloy of claim 23, wherein the magnesium alloy is free from Y.

31. The magnesium alloy of claim 23, wherein the magnesium alloy is free from rare earth elements.

32. The magnesium alloy of claim 23, wherein the magnesium alloy is extruded in two steps.

33. A medical device comprising the magnesium alloy of claim 23,
wherein the medical device is an implant.

34. A method of controlling the corrosion of a biodegradable magnesium alloy, comprising:
producing an alloy which comprises: 0.5-2 wt % Zn, 0.2-1 wt % Mn and 0.1-2 wt % Ca, and
controlling a corrosion rate by varying the content of Zn, Mn, and Ca.

35. A biodegradable implant comprising a body of a magnesium alloy,
wherein said magnesium alloy comprises Ca,
and wherein said magnesium alloy is coated with a coating which comprises Ca.

36. The biodegradable implant according to claim 35, wherein said coating is a calcium phosphate coating.

37. The biodegradable implant according to claim 35, wherein said magnesium alloy comprises at least 0.1 wt % Ca.

38. The biodegradable implant according to claim 35, wherein said magnesium alloy comprises
0.2-3 wt % Mn,
0.5-5 wt % Zn and/or
0.1-3 wt % Ca and/or
a ratio of Ca/Mn between 3:1 and 1:3 and/or
a ratio of Zn to the sum of Ca and Mn between 2:1 and 1:1.

39. The biodegradable implant according to claim 35, wherein said coating is a hydroxyapatite coating.

40. The biodegradable implant according to claim 35,
wherein said coating is applied by a method of plasma electrolytic oxidation and/or
wherein the magnesium alloy of the body gradually transitions to said coating.

41. The biodegradable implant according to claim 35,
wherein the magnesium content at least at the surface of the coating is more than 10 wt % and less than 70 wt %.

42. The biodegradable implant according to claim 35,
wherein the magnesium content at least at the surface of the coating is more than 30 wt % and less than 60 wt %.

43. The biodegradable implant according to claim 35, wherein the surface of said coating comprises 5-30 wt % Ca, 15-50 wt % 0 and/or 3-10 wt % P.

44. The biodegradable implant according to claim 35, wherein said coating comprises a ceramic-metal-matrix.

45. The biodegradable implant according to claim 35, wherein said coating has a thickness of 1 to 100 μm.

46. The biodegradable implant according to claim 35, wherein said coating has a thickness of 3 to 25 μm.

47. A method of producing a biodegradable implant,
wherein an implant body comprising a calcium containing magnesium alloy is coated with a calcium phosphate coating by using a plasma electrolytic oxidation method.

48. The method according to claim 47, wherein said plasma electrolytic oxidation process is performed in an electrolytic bath comprising calcium phosphate particles, in particular hydroxyapatite particles.

49. The method according to claim 47, wherein the electrolyte comprises water glass and/or a dissolved phosphate.

50. The method according to claim 47, wherein the electrolyte comprises water glass and sodium phosphate, each in an amount of at least 0.1%.
